(19) United States
(12) Patent Application Publication
(10) Pub. No.: US 2025/0186676 A1
(43) Pub. Date: Jun. 12, 2025

(54) NEGATIVE PRESSURE WOUND THERAPY ADVISORY SYSTEM, NEGATIVE PRESSURE WOUND THERAPY METHOD AND NON-TRANSITORY COMPUTER-READABLE MEDIA

(71) Applicant: NATIONAL CHENG KUNG UNIVERSITY, Tainan City (TW)

(72) Inventors: Chia-Ching WU, Tainan City (TW); Shyh-Hau WANG, Tainan City (TW); Da-Ming HUANG, Chiayi County (TW); Wan-Ching HUANG, New Taipei City (TW); Shin-Chen PAN, Tainan City (TW); Kuo-Shu HUNG, Taichung City (TW); I Ying LEE, Tainan City (TW)

(21) Appl. No.: 18/795,289

(22) Filed: Aug. 6, 2024

(30) Foreign Application Priority Data
Dec. 6, 2023 (TW) 112147464

(51) Int. Cl. A61M 1/00; G06T 7/00; G06T 7/10; G16H 30/40

(52) U.S. Cl. CPC A61M 1/96; G06T 7/0012; G06T 7/10; G16H 30/40; G06T 2207/30088; G06T 2207/30096

(57) ABSTRACT

A negative pressure therapy advisory system and a negative pressure therapy method by using the advisory system are provided. The method includes: an image preprocessing step and a dressing advisory step. The image preprocessing step includes: an image correction step, an image segmentation step and an image classification step to build a wound model, and to determine a wound injury degree, a wound shape, and blood vessels or anatomical tissue landmarks. The dressing advisory step provides a dressing geometry of a dressing shape in accordance with the wound model, in which the wound/dressing shape is a 2D shape or 3D shape. The method further includes: performing a negative pressure advisory step with respect to negative pressure wound therapy of the wound, or correcting colors of a wound pattern in the wound image by using a color reference calibrator. The system is configured to perform the above method.

### RELATED APPLICATIONS

This application claims priority to Taiwan Application Serial Number 112147464, filed on Dec. 6, 2023, which is herein incorporated by reference in its entirety.

### BACKGROUND

### Description of Related Art

Because negative pressure wound therapy (NPWT) has been highly successful in the promotion of wound closure and healing, NPWT can heal many wounds previously thought largely untreatable, for example decubitus and venous stasis ulcers to the lower extremities. Failure to close/heal these wounds, which have been present for several years, may cause necrotizing of the tissue. The necrotizing of the tissue may further cause amputation of the extremity. Use of NPWT has proven highly successful for healing/closing these wounds and avoids necrotizing of the tissue and amputation of the extremity.

### SUMMARY

Embodiments of the present invention provide a negative pressure therapy advisory system, a negative pressure therapy method and a non-transitory computer-readable media. The negative pressure therapy advisory system and the negative pressure therapy method can be applied to negative pressure wound therapy (NPWT) to increase the performance of the NPWT for wound healing.

In accordance with some embodiments of present invention, the negative pressure therapy advisory system includes: a memory and a processor. The memory is configured to store plural instructions. The processor is electrically connected to the memory to execute the instructions to perform steps of: performing a wound image processing step for a wound image of a wound to build a wound model of the wound; performing a dressing advisory step with respect to a dressing of the wound; and performing a negative pressure advisory step with respect to NPWT of the wound, or when the processor performs the image correction step, the processor performing: extracting a plurality of color pixel values of a plurality of color regions of a color checker pattern, and with respect to a plurality of color channels, calculating an average color pixel value of each of the color channels of the color regions to obtain a correction factor of each of the color channels, wherein the wound image includes the color checker pattern of the color checker and a wound pattern; and correcting colors of the wound pattern by using the correction factor of each of the color channels. The wound image processing step includes: performing an image preprocessing step, an image segmentation step, an image classification step. The image preprocessing step includes: performing an image correction step on the wound image by using a color checker to obtain a pixel size of the wound image in accordance with a scale pattern of the color checker, and to correct colors of the wound in the wound image. The image segmentation step includes: segmenting the wound image into a plurality of wound region images by using an image segmentation algorithm. The image classification step includes: classifying the wound region images by using a classification algorithm to build the wound model of the wound; and determining a wound injury degree, a wound shape, and a plurality of blood vessel or anatomical tissue positions in accordance with the wound model, wherein the wound shape is a two-dimensional shape or a three-dimensional shape. The dressing advisory step includes: providing a dressing shape of the dressing in accordance with the wound shape of the wound, wherein the wound shape is a two-dimensional shape or a three-dimensional shape; and providing a position for a suction head disposed on the dressing in accordance with the blood vessel or anatomical tissue positions. The negative pressure advisory step includes: adjusting an advisory value of negative pressure for NPWT in accordance with the wound injury degree of the wound.

In some embodiments, the dressing shape matches the wound shape to achieve an optimum negative pressure distribution.

In some embodiments, when the processor performs adjusting the advisory value of negative pressure for NPWT in accordance with the wound injury degree of the wound, the processor performs: comparing the wound injury degree of the wound image to a past wound injury degree of a past wound image to obtain a wound healing degree; decreasing the advisory value of negative pressure for NPWT when the wound healing degree represents wound healing; and changing or maintaining a negative pressure mode for NPWT, or increasing the advisory value of negative pressure for NPWT, when the wound healing degree represents aggravation of the wound.

In some embodiments, when the processor performs providing the position for the suction head disposed on the dressing in accordance with the blood vessel or anatomical tissue positions, the processor performs: calculating a potion for placing the suction head in accordance with the blood vessel or anatomical tissue positions of the wound, wherein the potion for placing the suction head provide promoting micro circulation of wound tissue and blood supply theory.

In some embodiments, the image segmentation step is performed by using a U-Net or R2U-Net algorithm.

In some embodiments, the image classification step is performed by using a fully convolutional networks (FCN) algorithm.

In some embodiments, the color checker pattern of the color checker includes a red color pattern, a blue color pattern and a green color pattern.

In some embodiments, the color checker pattern of the color checker further includes a yellow color pattern.

In accordance with some embodiments of present invention, the negative pressure wound therapy (NPWT) method includes: performing a wound image processing step by using an advisory system to build a wound model of a wound in accordance with a wound image of the wound; performing a dressing advisory step by using the advisory system; and performing a negative pressure advisory step by using the advisory system or the image correction step comprising: extracting a plurality of color pixel values of a plurality of color regions of a color checker pattern, and with respect to a plurality of color channels, calculating an average color pixel value of each of the color channels of the color regions to obtain a correction factor of each of the color channels, wherein the wound image includes the color checker pattern of the color checker and a wound pattern; and correcting colors of the wound pattern by using the correction factor of each of the color channels. The wound image processing step includes: performing an image preprocessing step, an image segmentation step, an image classification step. The image preprocessing step includes: performing an image correction step on the wound image by using a color checker to obtain a pixel size of the wound image in accordance with a scale pattern of the color checker, and to correct colors of the wound in the wound image. The image segmentation step includes: segmenting the wound image into a plurality of wound region images by using an image segmentation algorithm. The image classification step includes: classifying the wound region images by using a classification algorithm to build the wound model of the wound; and determining a wound injury degree, a wound shape, and a plurality of blood vessel or anatomical tissue positions in accordance with the wound model, wherein the wound shape is a two-dimensional shape or a three-dimensional shape. The dressing advisory step includes: providing a dressing shape of the dressing in accordance with the wound shape of the wound, wherein the wound shape is a two-dimensional shape or a three-dimensional shape; and providing a position for a suction head disposed on the dressing in accordance with the blood vessel or anatomical tissue positions. The negative pressure advisory step includes: adjusting an advisory value of negative pressure for NPWT in accordance with the wound injury degree of the wound.

In some embodiments, the dressing shape matches the wound shape to achieve an optimum negative pressure distribution.

In some embodiments, the step of adjusting the advisory value of negative pressure for NPWT in accordance with the wound injury degree of the wound includes: comparing the wound injury degree of the wound image to a past wound injury degree of a past wound image to obtain a wound healing degree; decreasing the advisory value of negative pressure for NPWT when the wound healing degree represents wound healing; and changing or maintaining a negative pressure mode for NPWT, or increasing the advisory value of negative pressure for NPWT, when the wound healing degree represents aggravation of the wound.

In some embodiments, the step of providing the position for the suction head disposed on the dressing in accordance with the blood vessel or anatomical tissue positions includes: calculating a potion for placing the suction head in accordance with the blood vessel or anatomical tissue positions of the wound, wherein the potion for placing the suction head provide promoting micro circulation of wound tissue and blood supply theory.

In some embodiments, the image segmentation step is performed by using a U-Net or R2U-Net algorithm.

In some embodiments, the image classification step is performed by using a fully convolutional networks (FCN) algorithm.

In some embodiments, the color checker pattern of the color checker includes a red color pattern, a blue color pattern and a green color pattern.

In some embodiments, the color checker pattern of the color checker further includes a yellow color pattern.

In some embodiments, embodiments of the present invention provide a non-transitory computer-readable media storing a program, and when a computer device load and execute the program, the computer device performs the above negative pressure wound therapy method.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic diagram showing a negative pressure therapy advisory system in accordance with embodiments of the present.

FIG. 2 is a schematic diagram showing a flow chart of a negative pressure wound therapy method by using the advisory system in accordance with the embodiments of the present invention.

FIG. 3 is a schematic diagram showing a flow chart of the wound image processing step in accordance with the embodiments of the present invention.

FIG. 4 is a schematic diagram showing a wound image in accordance with an embodiment of the present invention.

FIG. 5 is a schematic diagram showing a flowchart of fabrication of the dressing in accordance with embodiments of the present invention.

FIG. 6 is a schematic diagram showing a position for a suction head disposed on the dressing/wound in accordance with the blood vessel or anatomical tissue position.

### DETAILED DESCRIPTION

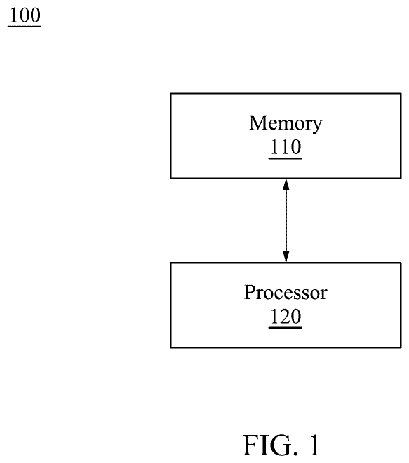

Referring to FIG. 1, FIG. 1 is a schematic diagram showing a negative pressure therapy advisory system 100 in accordance with embodiments of the present. The negative pressure therapy advisory system 100 includes a memory 110 and a processor 120. The memory 110 is configured to store plural instructions, and the processor 120 is electrically connected to the memory 110 to execute the instructions stored in the memory 110. In some embodiments, the negative pressure therapy advisory system 100 can be a personal computer, a notebook computer, or a tablet computer in practice, but embodiments of the present invention are not limited.

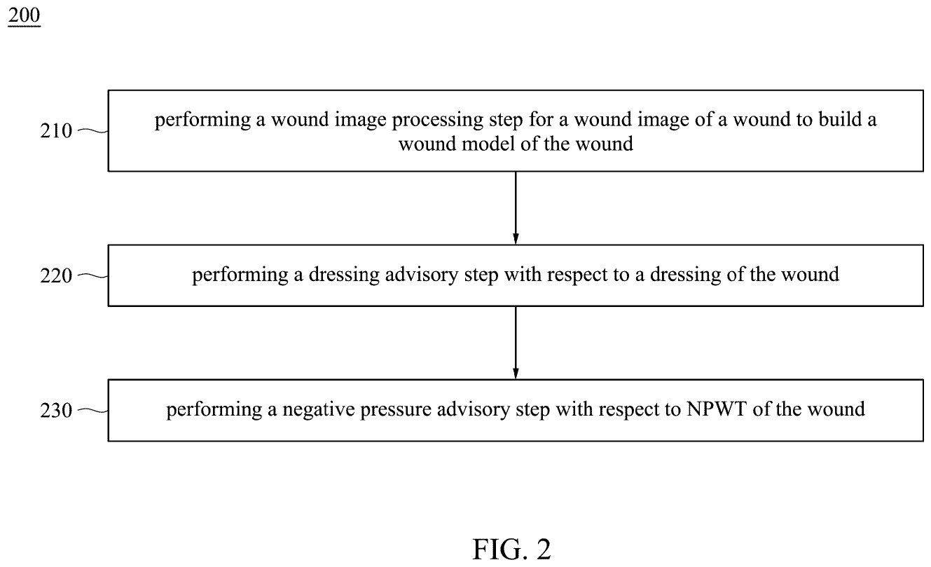

Referring to FIG. 2, FIG. 2 is a schematic diagram showing a flow chart of a negative pressure wound therapy (NPWT) method 200 by using the advisory system 100 in accordance with the embodiments of the present invention, in which the NPWT method 200 is adapted for the advisory system 100.

Specifically, after executing the instructions stored in the memory 110, the processor 120 can perform the NPWT method 200 to enable the advisory system 100 to provide better performance of NPWT for users.

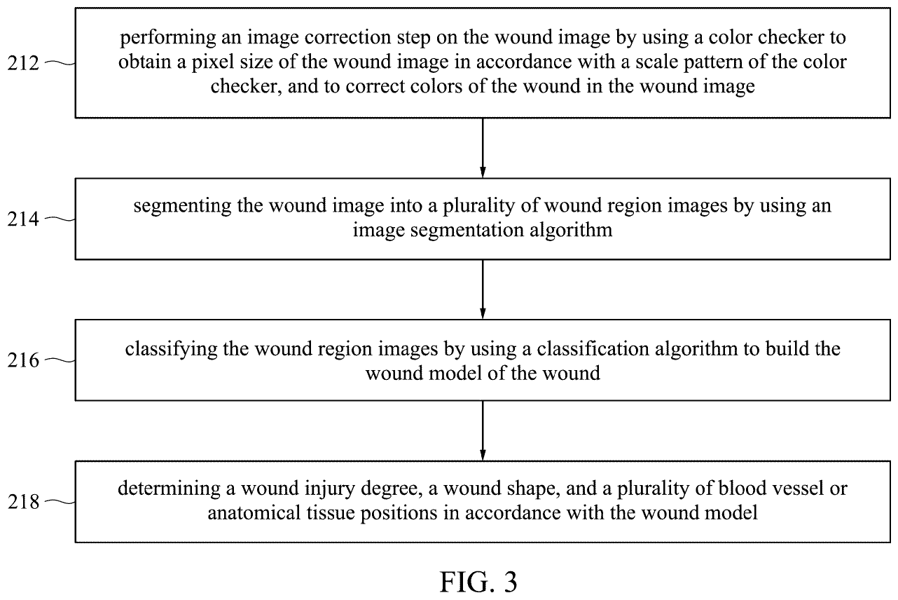

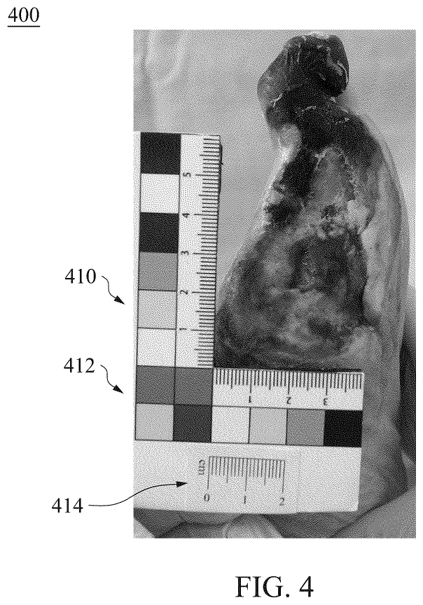

In the NPWT method 200, at first, step 210 is performed to use the advisory system 100 to perform a wound image processing step. The wound image processing step is performed to build a wound model of a wound in accordance with images of the wound. For example, the advisory system 100 is capable of receiving plural wound images from an external device, and building a wound model. Referring to FIG. 3, FIG. 3 is a schematic diagram showing a flow chart of the wound image processing step of step 210. In the embodiments of the present invention, the wound image processing step of step 210 includes steps 212-218, in which step 212 can be referred to as an image preprocessing step for preprocessing of the wound images; step 214 can be referred to as an image segmentation step for segmentation of the wound images; step 216 can be referred to as an image classification step for classification of the segmented wound images. Specifically, step 212 is performed to calculate a pixel size of the wound in the wound image in accordance with a scale pattern of a color checker, and to correct the color of the wound in the wound image in accordance with color patterns of the color checker, as shown in FIG. 4.

FIG. 4 is a schematic diagram showing a wound image 400 in accordance with one embodiment of the present invention. The wound image 400 includes a color checker 410, in which the color checker 410 includes color patterns 412 and a scale pattern 414. The scale pattern 414 is configured to present a unit scale, for example centimeter (cm). Therefore, the advisory system 100 is capable of calculating the pixel size of the wound in the wound image 400 in accordance with the unit scale of the scale pattern 414. For example, the pixel size of the wound in the wound image 400 may be 0.01 cm*0.01 cm. Further, the advisory system 100 is capable of using the colors of the color patterns 412 to correct the color of the wound in the wound image. Since the color of the wound represents the situation of the wound, the color correction of the embodiment of the present invention enables the wound image to more precisely represent different colors of the regions of the wound, for example black color, red color or pink color. In general, the black color of the wound represents necrotizing of the tissue of the wound; the red color of the wound represents proliferation of the tissue of the wound; the pink color of the wound represents closing of the wound.

Returning to FIG. 3, after step 212, step 214 is performed to use an image segmentation algorism to segment the wound image 400 into plural wound region images. In this embodiment, the segmentation of the wound image 400 is achieved by using an image segmentation algorithm (i.e., an image segmentation model). For example, at first, a data set including plural training samples of wound image is divided into a training set, a test set and a validation set, and a ratio of the training set, the test set and the validation set is 6:2:2. Then, the training set, the test set and the validation set are used to train the image segmentation model. In this embodiment, a U-Net or R2U-Net algorithm is used for building the image segmentation model. However, embodiments of the present invention are not limited thereto. In some embodiments, a DeconvNet algorithm, a SegNet algorithm or a RefineNet algorithm can be used for building the image segmentation model as well.

After step 214, step 216 is performed to use a classification algorithm to classify images of wound region images to build a wound model of the wound images of the wound. In this embodiment, the classification of the wound region images is achieved by using an image classification algorithm (i.e., a classification model). For example, feature extraction is performed on the wound images of the training samples. After the feature extraction, the image classification algorithm uses transition layers to decrease sizes of the feature patterns to reduce the space dimension of the patterns of the feature patterns. Then, up-sampling is used to recover sizes of outputted feature patterns to original patterns. Thereafter, weights of the fully connected layer of the image classification algorithm are converted to kernel, and new feature patterns are generated by computing the kernel and the feature patterns of a previous layer together, so that the fully connected layer can be converted to a convolution layer. Finally, the feature patterns are converted to a classification probability of each of the pixels, thereby obtaining a final classification result in accordance with the classification probability of each pixels, and building the classification model accordingly. In this embodiment, a fully convolutional networks (FCN) algorithm is used for building the classification model of the wound image. However, embodiments of the present invention are not limited thereto. In some embodiments, a Tensor Flow algorithm or a R2U-net algorithm can be used for building the classification model of the wound image as well. After the classification model is built, the classification model can be used to classify the wound region images obtained in step 214, and to build a wound model accordingly. For example, a can technology is used to scan the wound desired to be analyzed to build a 2D or 3D wound outline. Then, the wound outline and the classification model are combined (for example, combined in accordance with a position relationship of tissues) to obtain the wound model. Since the classification model is used in the wound model, the wound model is capable of identifying a wound state of each of the wound regions of the wound in accordance with the above classification model and the wound image of the wound image of the wound desired to be analyzed.

After step 216, step 218 is performed to determine a wound injury degree, a wound shape, and plural blood vessel or anatomical tissue positions in accordance with the wound model. For example, the advisory system 100 determines the wound injury degree in accordance with the color of each of the wound regions of the wound model. For another example, the advisory system 100 determines the wound shape in accordance with the wound outline of the wound model. For further another example, the advisory system 100 determines the blood vessel or anatomical tissue positions in accordance with the classification result of the wound model.

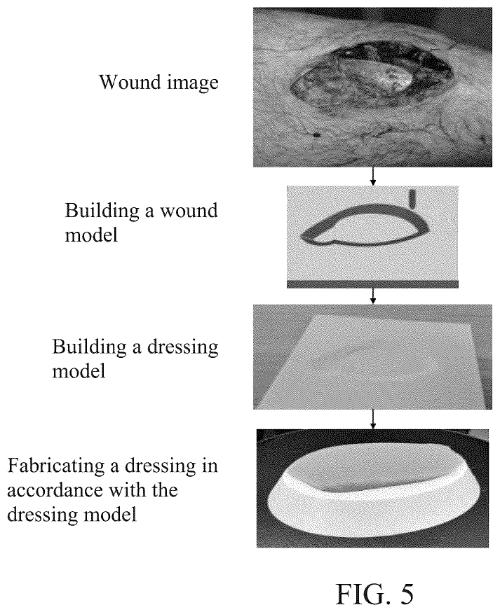

Returning to FIG. 2, after step 210, step 220 is performed for dressing advisory with respect to a dressing of the wound. In step 220, a dressing shape of the dressing is provided in accordance with the wound shape of the wound, so that the dressing matching the wound can be fabricated in accordance with dressing shape. Referring to FIG. 5, FIG. 5 is a schematic diagram showing a flowchart of fabrication of the dressing in accordance with embodiments of the present invention. As shown in FIG. 5, after building the wound model of the wound in accordance with the wound image, a dressing mold is fabricated in accordance with the wound model. The dressing mold can be fabricated by using a 3D printing technology or a mold stamping technology to enable the dressing mold to have an outline corresponding to the wound model. Then, the dressing mold can be used to fabricate the dressing having a shape matching the wound shape, and thus the fabricated dressing matches the wound. The dressing matching the wound is capable of achieving an optimum negative pressure distribution when NPWT is performed and enhancing the performance of NPWT. In this embodiment, the dressing is a foam dressing, but embodiments of the present invention are not limited thereto. In some embodiments, the dressing is a silicone dressing.

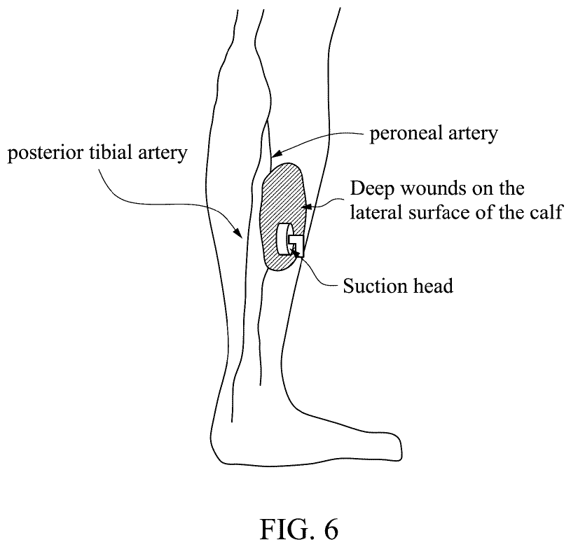

In some embodiments, the dressing advisory step provides a position for a suction head disposed on the dressing/wound in accordance with the blood vessel or anatomical tissue position. For example, as shown in FIG. 6, when a calf has a deep wound on the lateral surface and NPWT is required for healing the deep wound, the advisory system 100 can be used to calculate the position for placing the suction head in accordance with promoting micro circulation of wound tissue and blood supply theory. For example, the suction head can be placed at a position far away from the posterior tibial artery and the peroneal artery of the calf to promote micro circulation and blood supply of the deep wound on the lateral surface of the calf.

Returning to FIG. 2, the NPWT method 200 may further includes step 230 to provide an advisory value of negative pressure for NPWT in accordance with the wound injury degree of the wound. In some embodiments, the NPWT can be performed in various modes, for example a continuous mode, an intermittent mode, a dynamic mode and a cycling mode. The pressure value of NPWT is in a range from −25 mm Hg to −150 mm Hg. In other words, step 230 provides the advisory value of negative pressure ranged from −25 mm Hg to −150 mm Hg in accordance with the wound injury degree of the wound. In some embodiments, the wound injury degree of the wound image is compared to a past wound injury degree of a past wound image to obtain a wound healing degree. When the wound healing degree represents healing of the wound, the advisory value of negative pressure for NPWT is decreased. When the wound healing degree represents aggravation of the wound, the negative pressure mode for NPWT is changed or maintained, or the advisory value of negative pressure for NPWT is increased. For example, when the color of the wound is changed from a red color to a pink color, it is determined that the wound healing degree represents healing of the wound, and the advisory value of negative pressure for NPWT is decreased (for example, decreased from −140 mm Hg to −70 mm Hg). For another example, when the color of the wound is changed from a red color to a yellow green color, it is determined that the wound healing degree represents aggravation of the wound, and the advisory value of negative pressure for NPWT is increased (for example, decreased from −120 mm Hg to −150 mm Hg), or the advisory system 100 may provide an advice that the negative pressure mode is required to be changed for NPWT.

In some embodiments, the image correction step may calculate a correction factor to correct the colors of the patterns of the wound in the wound image. For example, plural color pixel values of color regions of the color checker 410 (for example, color patterns 412) are extracted. In this embodiment, the color patterns 412 include a red color pattern, a blue color pattern, a green color pattern and a yellow color pattern. However, embodiments of the present invention are not limited thereto. For example, the yellow color pattern in the color patterns 412 can be omitted. For another example, the color patterns 412 may have other color patterns replacing the above color patterns. Specifically, the red color pattern, the blue color pattern and the green color pattern can be replaced by a cyan color pattern, a magenta color pattern and a yellow color pattern.

After the color values of the red color pattern, the blue color pattern and the green color pattern are extracted, for the color channels of the red color, the blue color and the green color, an average color pixel value of each of the color channels of the red color, the blue color and the green color is calculated, thereby obtaining a correction factor of each of the color channels of the red color, the blue color and the green color. Therefore, the correction factor of each of the color channels can be used to correct the colors of the wound. Specifically, when the color correction is performed, the correction factor of each of the color channels is used as a reference value of the color of the wound and compared to one of real three primary colors to obtain a color correction value. For example, for the red color, an average color pixel value of the red color channel of the color patterns 412 is calculated, thereby obtaining a correction factor of the red color channel. Then, the correction factor of the red color channel is compared to the value of the red color of the real three primary colors, to obtain a difference value of the red color. The difference value of the red color can be used as a correction value for the red color channel of the wound image, and used to adjust the value of the red color of the red color channel of the wound image.

It can be understood from the above descriptions that the advisory system 100 and the NPWT method 200 using the advisory system 100 build a wound model for dressing advisory and negative pressure value advisory, thereby enhancing the performance of NPWT. Specifically, the embodiments of the present invention are capable of achieving an optimum negative pressure distribution and promoting micro circulation of wound tissue and blood supply, thereby enhancing the performance of NPWT. Furthermore, the embodiments of the present invention is capable of adjusting advisory value of negative pressure/changing negative pressure mode to enable NPWT to react on time with respect to the change of the situation of the wound, thereby healing the wound more effectively.

In some embodiments, the NPWT method 200 can be embodied as a non-transitory computer-readable media storing a program. When a computer device load and execute the program, the computer device performs the NPWT method 200.

While the disclosure has been described by way of example(s) and in terms of the preferred embodiment(s), it is to be understood that the disclosure is not limited thereto. Those skilled in the art may make various changes, substitutions, and alterations herein without departing from the spirit and scope of the present disclosure. In view of the foregoing, it is intended that the present invention cover modifications and variations of this invention provided they fall within the scope of the following claims.

What is claimed is:

1. A negative pressure wound therapy (NPWT) advisory system comprising:
a memory configured to store a plurality of instructions; and
a processor electrically connected to the memory, wherein the processor is configured to execute the instructions to perform steps of:
performing a wound image processing step for a wound image of a wound to build a wound model of the wound, wherein the wound image processing step comprises:
performing an image preprocessing step comprising:
performing an image correction step on the wound image by using a color checker to obtain a pixel size of the wound image in accordance with a scale pattern of the color checker, and to correct colors of the wound in the wound image;
performing an image segmentation step comprising:
segmenting the wound image into a plurality of wound region images by using an image segmentation algorithm;
performing an image classification step comprising:
classifying the wound region images by using a classification algorithm to build the wound model of the wound; and
determining a wound injury degree, a wound shape, and a plurality of blood vessel or anatomical tissue positions in accordance with the wound model, wherein the wound shape is a two-dimensional shape or a three-dimensional shape;
performing a dressing advisory step with respect to a dressing of the wound, wherein the dressing advisory step comprises:
providing a dressing shape of the dressing in accordance with the wound shape of the wound, wherein the wound shape is a two-dimensional shape or a three-dimensional shape; and
providing a position for a suction head disposed on the dressing in accordance with the blood vessel or anatomical tissue positions; and
performing a negative pressure advisory step with respect to NPWT of the wound, or when the processor performs the image correction step, the processor performing:
extracting a plurality of color pixel values of a plurality of color regions of a color checker pattern, and with respect to a plurality of color channels, calculating an average color pixel value of each of the color channels of the color regions to obtain a correction factor of each of the color channels, wherein the wound image comprises the color checker pattern of the color checker and a wound pattern; and
correcting colors of the wound pattern by using the correction factor of each of the color channels;
wherein the negative pressure advisory step comprises:
adjusting an advisory value of negative pressure for NPWT in accordance with the wound injury degree of the wound.

2. The negative pressure wound therapy advisory system of claim 1, wherein the dressing shape matches the wound shape to achieve an optimum negative pressure distribution.

3. The negative pressure wound therapy advisory system of claim 1, wherein when the processor performs adjusting the advisory value of negative pressure for NPWT in accordance with the wound injury degree of the wound, the processor performs:
comparing the wound injury degree of the wound image to a past wound injury degree of a past wound image to obtain a wound healing degree;
decreasing the advisory value of negative pressure for NPWT when the wound healing degree represents wound healing; and
changing or maintaining a negative pressure mode for NPWT, or increasing the advisory value of negative pressure for NPWT, when the wound healing degree represents aggravation of the wound.

4. The negative pressure wound therapy advisory system of claim 1, wherein when the processor performs providing the position for the suction head disposed on the dressing in accordance with the blood vessel or anatomical tissue positions, the processor performs:
calculating a potion for placing the suction head in accordance with the blood vessel or anatomical tissue positions of the wound, wherein the potion for placing the suction head provide promoting micro circulation of wound tissue and blood supply theory.

5. The negative pressure wound therapy advisory system of claim 1, wherein the image segmentation step is performed by using a U-Net or R2U-Net algorithm.

6. The negative pressure wound therapy advisory system of claim 1, wherein the image classification step is performed by using a fully convolutional networks (FCN) algorithm.

7. The negative pressure wound therapy advisory system of claim 1, wherein the color checker pattern of the color checker comprises a red color pattern, a blue color pattern and a green color pattern.

8. The negative pressure wound therapy advisory system of claim 7, wherein the color checker pattern of the color checker further comprises a yellow color pattern.

9. A negative pressure wound therapy (NPWT) method by using an advisory system, comprising:
performing a wound image processing step by using an advisory system to build a wound model of a wound in accordance with a wound image of the wound, wherein the wound image processing step comprises:
performing an image preprocessing step comprising:
performing an image correction step on the wound image by using a color checker to obtain a pixel size of the wound image in accordance with a scale pattern of the color checker, and to correct colors of the wound in the wound image;
performing an image segmentation step comprising:
segmenting the wound image into a plurality of wound region images by using an image segmentation algorithm;
performing an image classification step comprising:
classifying the wound region images by using a classification algorithm to build the wound model of the wound; and
determining a wound injury degree, a wound shape, and a plurality of blood vessel or anatomical tissue positions in accordance with the wound model, wherein the wound shape is a two-dimensional shape or a three-dimensional shape;
performing a dressing advisory step by using the advisory system, wherein the dressing advisory step comprises:
providing a dressing shape of a dressing in accordance with the wound shape of the wound, wherein the wound shape is a two-dimensional shape or a three-dimensional shape; and
providing a position for a suction head disposed on the dressing in accordance with the blood vessel or anatomical tissue positions; and
performing a negative pressure advisory step by using the advisory system, or the image correction step comprising:
extracting a plurality of color pixel values of a plurality of color regions of a color checker pattern, and with respect to a plurality of color channels, calculating an average color pixel value of each of the color channels of the color regions to obtain a correction factor of each of the color channels, wherein the wound image comprises the color checker pattern of the color checker and a wound pattern; and
correcting colors of the wound pattern by using the correction factor of each of the color channels;
wherein the negative pressure advisory step comprises:
adjusting an advisory value of negative pressure for NPWT in accordance with the wound injury degree of the wound.

10. The negative pressure wound therapy method of claim 9, wherein the dressing shape matches the wound shape to achieve an optimum negative pressure distribution.

11. The negative pressure wound therapy method of claim 9, wherein adjusting the advisory value of negative pressure for NPWT in accordance with the wound injury degree of the wound comprises:
comparing the wound injury degree of the wound image to a past wound injury degree of a past wound image to obtain a wound healing degree;
decreasing the advisory value of negative pressure for NPWT when the wound healing degree represents wound healing;
changing or maintaining a negative pressure mode for NPWT, or increasing the advisory value of negative pressure for NPWT, when the wound healing degree represents aggravation of the wound.

12. The negative pressure wound therapy method of claim 9, wherein providing the position for the suction head disposed on the dressing in accordance with the blood vessel or anatomical tissue positions comprises:
calculating a potion for placing the suction head in accordance with the blood vessel or anatomical tissue positions of the wound, wherein the potion for placing the suction head provide promoting micro circulation of wound tissue and blood supply theory.

13. The negative pressure wound therapy method of claim 9, wherein the image segmentation step is performed by using a U-Net or R2U-Net algorithm.

14. The negative pressure wound therapy method of claim 9, wherein the image classification step is performed by using a fully convolutional networks (FCN) algorithm.

15. The negative pressure wound therapy method of claim 9, wherein the color checker pattern of the color checker comprises a red color pattern, a blue color pattern and a green color pattern.

16. The negative pressure wound therapy method of claim 15, wherein the color checker pattern of the color checker further comprises a yellow color pattern.

17. A non-transitory computer-readable media storing a program, wherein when a computer device load and execute the program, the computer device performs the method of claim 9.
